(19) United States
(12) Patent Application Publication
(10) Pub. No.: US 2013/0178682 A1
(43) Pub. Date: Jul. 11, 2013

(54) NON-OXIDATIVE DEHYDROGENATIVE PROCESS

(71) Applicants: Lin Luo, Sugar Land, TX (US); Devon C. Rosenfeld, Houston, TX (US)

(72) Inventors: Lin Luo, Sugar Land, TX (US); Devon C. Rosenfeld, Houston, TX (US)

(73) Assignee: Dow Global Technologies LLC, Midland, MI (US)

(21) Appl. No.: 13/814,583

(22) PCT Filed: Aug. 24, 2011
(86) PCT No.: PCT/US2011/048934

§ 371 Date: Feb. 6, 2013

Related U.S. Application Data
(60) Provisional application No. 61/388,087, filed on Sep. 30, 2010.

(51) Int. Cl. C07C 5/333

(52) U.S. Cl. CPC C07C 5/3337
USPC 585/660

(57) ABSTRACT

Use a supported catalyst that includes a catalyst support and a combination of nickel, gallium, and, optionally, one or more of an alkali metal, an alkaline earth metal and a lanthanide to effect non-oxidative dehydrogenation of an oxygen-free gaseous stream that comprises an alkane such as ethane, propane or butane to a product stream that comprises a corresponding alkene such as propylene where the alkane is propane.

This application is a non-provisional application claiming priority from the U.S. Provisional Patent Application No. 61/388,087, filed on Sep. 30, 2010, entitled “IMPROVED NON-OXIDATIVE DEHYDROGENATIVE PROCESS” the teachings of which are incorporated by reference herein, as if reproduced in full hereinbelow.

This invention relates generally to an improved process for non-oxidatively dehydrogenating an alkane to its corresponding alkene, particularly for dehydrogenating ethane to ethylene, propane to propylene or butane to butylene, in a circulating fluidized bed reactor, preferably a circulating fluidized bed reactor based upon a riser reactor, and more particularly to active, stable catalysts used in the improved process.

Alkane dehydrogenation (ADH) in general and propane dehydrogenation (PDH) in particular, is limited by thermodynamics. Increasing alkane conversion, especially propane conversion through a rise in reaction temperature, however, leads to more rapid catalyst deactivation and higher rates of coke formation than one sees without the rise in reaction temperature. In addition, efforts to raise reaction temperature give rise to a difficulty in supplying more heat to the apparatus used to effect alkane (e.g. propane) dehydrogenation.

Conventional ADH or PDH catalysts typically require a high loading (e.g. 10 percent by weight (wt %) or more based upon total catalyst weight) of chromium oxide (as Cr₂O₃), a material that prompts some environmental concerns, or platinum (Pt) a very expensive noble metal. Pt-based catalysts, aside from being very expensive, may undergo deactivation due at least in part to one or more of Pt loss, attrition, Pt metal sintering and blocking of active sites with coke.

U.S. Pat. No. 4,056,576 (Gregory et al.) discloses a process for producing unsaturated hydrocarbons by dehydrogenating saturated hydrocarbons that contain from three to eight carbon atoms (C₃-C₈) in the presence of a supported gallium (Ga) catalyst. Suitable supports include aluminas such as eta-alumina, gamma alumina and boehmite, aluminas and silicas with or without surface hydroxyl groups that may be exchanged by ions of metals selected from Ga, aluminum (Al), iron (Fe) and nickel (Ni), activated carbon and refractory gallium oxide. The catalyst may contain other metals such as palladium (Pd), Pt, indium (In), thallium (Tl), germanium (Ge), chromium (Cr), tin (Sn) and zinc (Zn).

U.S. Pat. No. 4,125,565 (Antos) teaches dehydrogenation of hydrocarbons using a catalytic composite comprising a platinum group component (Pt, Pd, iridium (Ir), osmium (Os), ruthenium (Ru), rhodium (Rh) or mixtures thereof), a nickel (Ni) component and a bismuth (Bi) component on a porous carrier. The composite may also comprise an alkali metal or alkaline earth metal component.

U.S. Pat. No. 4,914,075 (Bricker, J. C. et al.) discloses a hydrocarbon dehydrogenation catalyst comprising a Group VIII noble metal (Pt, Pd, Ir, Rh, Os, Ru or mixtures thereof), a second component selected from tin (Sn), germanium (Ge), lead (Pb), indium (In), Ga, thallium (Tl) or mixtures thereof, and a third component selected from alkali metals, alkaline earth metals or mixtures thereof. The components are supported on theta-alumina. The catalyst contacts a dehydrogenatable hydrocarbon in a fixed, moving or fluidized bed system. See also U.S. Pat. No. 6,756,340 (Voskoboynikov et al.) for similar catalyst compositions.

U.S. Pat. No. 7,235,706 (Iezzi et al.) provides a process for preparing light olefins by dehydrogenating corresponding paraffins in a fluidized bed type reactor and regenerator. The catalyst comprises Ga (as Ga₂O₃), Pt, an alkaline earth metal or alkali metal and silica on alumina. The alumina is in delta plus theta phase, theta plus alpha phase, or delta plus theta plus alpha mixed phase.

Patent Cooperation Treaty Application (WO) 2005/077867 (Pretz et al.) discusses use of a circulating fluidized bed (CFB) process for dehydrogenating a paraffinic hydrocarbon compound such as an alkane (e.g. propane) to its corresponding olefin (propylene in the case of propane as the alkane) or an alkylaromatic compound (e.g. ethylbenzene) to its corresponding aromatic compound (styrene in the case of ethylbenzene as the alkylaromatic compound) using a catalyst comprising Ga on an alumina or alumina-silica support. The catalyst may also comprise at least one alkali metal or alkaline earth metal along with at least one of Pt and manganese (Mn). The CFB process suitably employs a tubular, cylindrical riser reactor connected to a product gas exit line at its upper end and a fresh or regenerated catalyst line at its lower end. The process uses a separation device to separate spent or deactivated catalyst from product gas and sends the spent or deactivated catalyst to a regenerator. Regenerated catalyst flows back to the reactor via the fresh or regenerated catalyst line. Combustion air and, optionally, supplemental fuel provide heat of reaction and sensible heat as needed to effect the dehydrogenation reaction.

U.S. Pat. No. 6,031,143 (Buonomo et al.) teaches a process for producing styrene via dehydrogenation that uses a catalyst based on Ga and Pt on alumina in delta or theta phase or in a mixture of delta and theta, theta and alpha or delta, theta and alpha phases, modified with silica, and having a BET surface area less than 100 m²/g.

A case family that includes U.S. Pat. No. 7,498,289 (Liu), U.S. Pat. No. 7,227,049 (Liu), U.S. Pat. No. 6,777,371 (Liu), U.S. Pat. No. 6,417,422 (Liu), U.S. Pat. No. 6,355,854 (Liu) and U.S. Pat. No. 6,436,871 (Liu), provides teachings about catalysts and methods for alkane (e.g. propane) oxydehydrogenation. The catalysts comprise (i) nickel (Ni) or a nickel-containing compound, and (ii) at least one of titanium (Ti), tantalum (Ta), niobium (Nb), hafnium (Hf), tungsten (W), yttrium (Y), zinc (Zn), zirconium (Zr) or aluminum (Al), or a compound containing one or more of such elements. Preferred catalysts include a catalyst support. The methods require a co-feed of oxygen with the alkane at a temperature of from 250 degrees Celsius (° C.) to 350° C. U.S. Pat. No. 6,436,871 specifies that the catalytic metals be oxides. See also related WO 00/48971.

U.S. Pat. No. 5,639,929 (Bharadwaj et al.) relates to oxidative dehydrogenation of hydrocarbons by contacting a two to six carbon (C₂-C₆) alkane with an oxygen-containing gas in a fluidized catalyst bed of platinum (Pt), rhodium (Rh), Ni, or platinum-gold (Pt-Au) supported on alpha-alumina or zirconia. See also related PCT Application WO 96/33149.

U.S. Pat. No. 5,254,779 (Mazzocchia et al.) teaches a nickel-molybdenum oxide catalyst and its use in oxidative dehydrogenation of propane at a temperature of from 400° C. to 700° C. See also related EP 379433.

U.S. Pat. No. 4,751,342 (Kimble) discloses a process for dehydrogenating C₂-C₁₂ paraffins to olefins in the presence of free oxygen, steam and a catalyst that comprises Ni, phosphorous (P), tin (Sn), oxygen (O) and, optionally, alkali metal. The process requires that ammonia (NH₃) be present in a ratio of NH₃:paraffin of at least 1:100.

In some aspects, this invention is an improved non-oxidative dehydrogenation process for catalytically converting an oxygen-free gaseous feed stream that comprises an alkane selected from ethane, propane and butane to a product stream that comprises an alkene that corresponds to the alkane (ethylene from ethane, propylene from propane, butylene from butane) by contacting the gaseous feed stream with a dehydrogenation catalyst at reaction temperature and in concurrent flow through a dehydrogenation reactor for an average contact time between the gaseous stream and the catalyst that is less than or equal to 20 seconds, wherein the improvement comprises using a supported catalyst that consists essentially of nickel, gallium and, optionally, one or more elements selected from Group VII noble metals, alkali metals, alkaline earth metals and lanthanides, on a catalyst support.

The use of Ni allows one to effect PDH at a lower cost than when using Pt as a catalytic metal. It also allows one to avoid environmental concerns noted above when using Cr as a catalytic metal.

The reaction temperature preferably lies within a range of from 570° Celsius to 750° Celsius. Pressure within the reactor preferably lies within a range of from 50.7 kilopascals (KPa) to 2 megapascals (MPa), more preferably from 101 KPa to 304 KPa.

In a riser reactor, inject an alkane (e.g. ethane, propane or butane) feedstream into a riser reactor and mix the feedstream with hot catalyst to form a combined stream that moves upward in the riser reactor with dehydrogenation of the feedstream occurring concurrent with such upward movement. The catalyst and gaseous products exit the riser reactor and enter a separation zone or apparatus such as a cyclone. After separation, strip the catalyst with gaseous nitrogen (N₂) before sending it to a regenerator. In the regenerator, remove coke from the catalyst, heat it to a target temperature (e.g. 600 degrees Celsius (° C.) to 800° C.), optionally strip with an inert gas, and then circulate the heated catalyst back to the riser reactor. The combination of riser reactor, separation zone and regenerator described herein may also be referred to as a “circulating fluidized bed reactor”.

The catalyst contains a mid or late transition metal, sometimes referred to as a Group VIII (also known as Group VIIIA or Groups 8, 9 and 10 depending upon which notation or version of The Periodic Table of The Elements one uses) metal. Such metals include iron (Fe), ruthenium (Ru), osmium (Os), cobalt (Co), rhodium (Rh), iridium (Ir), nickel (Ni) and palladium (Pd). In addition to the mid or late transition metal, preferably Ni, the catalyst must contain gallium (Ga) and it optionally includes one or more elements selected from Group VIII noble metals (other than the mid or late transition metal, alkali metals, alkaline earth metals and lanthanides). Group VIII noble metals include Rh, Ir, platinum (Pt), Pd, Ru and Os, with Rh, Ir, Pt and Pd being preferred when a Group VIII noble metal is present. When a Group VIII noble metal is present, it is an element that differs from the mid or late transition metal. By way of example, Pt is a preferred Group VIII noble metal when the mid or late transition metal (also designated broadly as Group VIII) is Ni. All of such metals are dispersed on a catalyst support, preferably an alumina support. The alumina support optionally further comprises silica in an amount within a range of from 0 wt % (when no silica is present) to 2 wt %, with 1.5 wt % silica yielding very satisfactory results and each wt % being based upon total support weight. The alumina support may comprise one or more of the following alumina phases: alpha, delta, gamma, and theta. The catalyst preferably comprises, consists essentially of or consists of at least one of nickel, ruthenium, cobalt or iron, and more preferably nickel, in conjunction with gallium and, optionally, an alkali metal selected from cesium, lithium and potassium, preferably potassium.

The catalyst support has a Brunauer, Emmet and Teller (BET) surface area that ranges from 50 square meters per gram (m²/g) to 150 m²/g, preferably from 65 m²/g to 125 m²/g, with very satisfactory results being obtained with a surface area of 70 m²/g.

The mid or late transition metal, preferably Ni, is present in an amount of from 10 parts by weight per million parts by weight of supported catalyst (ppm) to 500 ppm, preferably from 25 ppm to 500 ppm, and more preferably from 50 ppm to 200 ppm. Ga is present in an amount within a range of from 0.1 weight percent (wt), based upon weight of supported catalyst, to 5 wt %, preferably from 0.2 wt % to 2.0 wt %. The alkali metal, alkaline earth metal or lanthanide, preferably alkali metal and more preferably cesium, lithium or potassium is present in an amount of from 0 wt % to 5 wt %, preferably from 0.05 wt % to 1 wt %. When the catalyst includes a Group VIII noble metal, the Group VIII metal noble metal (e.g., Pt) replaces a portion, but not all, of the mid or late transition metal (e.g. Ni). A ratio of mid or late transition metal to Group VIII noble metal preferably ranges from 100:0 to less than 0:100. For avoidance of doubt, a 0 wt % loading means that the catalyst does not contain any of the optional alkali metal, alkaline earth metal or lanthanide.

Catalyst preparation may occur by way of any conventional technique such as aqueous incipient wetness which is used below in illustrative examples. Catalyst preparation includes wetting a dried catalyst support with a solution of one or more catalytic metal precursors, drying the wetted catalyst support and thereafter calcining the wetted and then dried catalyst support. Calcination preferably occurs at a temperature of less than 750° C., e.g. at a temperature of approximately 600° C.

The circulating fluidized bed reactor described hereinabove allows for a short contact time between the alkane feedstream and the heated catalyst. The contact time is preferably less than 20 seconds and more preferably from two seconds to five seconds,. The short contact time minimizes secondary reactions such as product (alkene) decomposition and coke formation. The catalyst has a short residence time within the reactor, typically on the order of from 0.5 seconds to 40 seconds, which enhances catalyst activity preservation, relative to that experienced with much longer residence times such as nine to 15 minutes.

In succeeding paragraphs, “Ex” with an Arabic numeral represents examples that illustrate aspects of this invention and “CEx” with a capital letter designates comparative examples.

### Ex 1

In an oven operating at a set point temperature of 350° C., pre-dry a 300 gram (g) aliquot of alumina/silica (1.5 wt % silica based upon combined weight of alumina and silica) (SIRALOX™ 1.5/70, commercially available from Sasol for two (2) hours (hr). Remove the aliquot from the oven and allow it to cool to ambient temperature (nominally 25° C.). Place 60 g of the pre-dried aliquot into a 600 milliliter (mL) beaker. Into a 100 mL beaker equipped with a magnetic stir bar, charge 0.016 g of nickel nitrate hexahydrate (Ni(NO₃)₂(H₂O)₆), 0.388 g of potassium nitrate (KNO₃), 5.01 g of gallium nitrate hexahydrate (Ga(NO₃)₃(H₂O)₆), and 13.2 mL of deionized (DI) water. Stir contents of the 100 mL beaker until the metal nitrates dissolve to form a visually uniform solution. Add, with stirring, small aliquots, less than 1 mL of the contents of the 100 mL beaker to the pre-dried aliquot in the 600 mL beaker to effect dispersion of the metal nitrates into and onto the pre-dried aliquot. Rinse the 100 mL beaker with approximately 2 mL DI water, then add the rinse water to the 600 mL beaker with continued stiffing to provide a wetted support. Discontinue stiffing and place the 600 mL beaker in a fume hood overnight. Remove the beaker from the fume hood, and dry its contents at 120° C. for four hours before calcining the contents for four hours at 600° C. The calcined material (catalyst) has a nickel content of 50 parts by weight per million parts by weight (ppm) of calcined material, a potassium content of 0.25 wt % and a gallium content of 1.6 wt %, each wt % being based upon calcined material weight. Remove 10 g of the calcined beaker contents and subject it to a second calcination at 750° C. for four hours.

Evaluate performance of the catalyst calcined at 600° C. using a plug flow reactor modified to test catalysts under short contact time between reactant and catalysts by exposing 0.5 g of the catalyst, mixed with 1 g of silicon carbide (SiC) as a diluent, to a feedstream that comprises 56.4 mole percent (mol %) propane. 40.7 mol % helium and 2.9 mol % nitrogen, each mol % being based upon combined moles of propane, helium and nitrogen and flows at a flow rate of 30 reciprocal hours (hr⁻¹), a reaction pressure of one atmosphere (atm) (101.3 kilopascals (KPa) and a reaction temperature of 580° C. Regenerate the catalyst by exposing it to a temperature of 700° C. with an air flow of 150 standard cubic centimeters per minute for a period of 900 seconds.

Gas chromatography was employed for effluent composition analysis. Sampling for GC analysis was conducted after 20 seconds of catalyst time on stream (TOS or exposure of catalyst to the feedstream at reaction conditions as specified above) and again after 10 minutes of catalyst TOS. The compounds analyzed include methane, ethane, ethylene, propane, propylene, C₄s (butane and butenes), C₅s, C₆s, N₂ internal standard, CO and CO₂.

Calculate alkane conversion and selectivity to its corresponding alkene (e.g. ethane to ethylene, propane to propylene or butane to butylene) as follows:
Alkane conversion=Total moles of alkane equivalents in product per min/(Moles of alkane in effluent per min+Total moles of alkene equivalents in product per min)
Selectivity for Alkene=Moles alkane equivalents in alkene per min/Total moles alkane equivalents in products per min

### Ex 2, Ex 3 and CEx A through CEx F

Replicate Ex 1 with calcination only at 600° C., but change the nickel content to 0 ppm for CEx A, 10 ppm for CEx B, 25 ppm for CEx C, 100 ppm for Ex 2, 200 ppm for Ex 3, 500 ppm for CEx D, 1000 ppm for CEx E and 5000 ppm for CEx F. For a 20 second TOS, see Table I below for propane conversion, propylene selectivity and propylene yield data.

### CEx G

Replicate Ex 3, but eliminate the gallium. Summarize results in Table 1 below.

### CEx H through CEx M

Replicate Ex 1, but change the catalyst nickel content as shown in Table 1 below and use catalyst subjected to the second calcination at 750° C. as detailed above. Summarize test results in Table 1 below.

TABLE 1
Catalyst Performance Data

| | | Ga | Propane | Selectivity to | Propylene |
|---|---|---|---|---|---|
| Ex/CEx | ppm Ni | (wt %) | Conversion (%) | Propylene (%) | Yield (%) |
| 1 | 50 | 1.6 | 37 | 99.5 | 36.3 |
| 2 | 100 | 1.6 | 32 | 99.5 | 32.2 |
| 3 | 200 | 1.6 | 32 | 99.5 | 31.6 |
| A | 0 | 1.6 | 22 | 99.3 | 22.3 |
| B | 10 | 1.6 | 12 | 98.7 | 11.7 |
| C | 25 | 1.6 | 17 | 99.3 | 16.5 |
| D | 500 | 1.6 | 21 | 99.3 | 20.6 |
| E | 1000 | 1.6 | 16 | 99.2 | 16.2 |
| F | 5000 | 1.6 | 11 | 98.2 | 10.4 |
| G | 200 | 0 | 2 | 91.9 | 1.8 |
| H | 0 | 1.6 | 25 | 99.5 | 25.1 |
| I | 50 | 1.6 | 13 | 98.7 | 13.2 |
| J | 100 | 1.6 | 17 | 99.1 | 16.8 |
| K | 200 | 1.6 | 22 | 99.4 | 22.1 |
| L | 500 | 1.6 | 20 | 99.3 | 19.5 |
| M | 5000 | 1.6 | 11 | 98.7 | 11.1 |

A comparison of Ex 1 through Ex 3 with CEx A through CEx F shows that under the catalyst preparation and reaction conditions specified in those examples and comparative examples, a nickel loading of more than 25 ppm (CEx C) and less than 500 ppm (CEx D) provides greater propane conversion than nickel loadings of either 0 ppm (CEx A) or 500 ppm (CEx D) with a nickel loading of 50 ppm (Ex 1) providing the best propane conversion. A comparison of Ex 3 and CEx H suggests that the catalyst must contain three metals (gallium, nickel and potassium) rather than two metals (nickel and potassium). CEx H through CEx M show that the second calcination at 750° C. adversely affects propane conversion at all but the highest nickel loading of 5000 ppm. CEx H through CEx M therefore suggest that the catalyst be calcined at a temperature of less than 750° C., with the 600° C. calcination of Ex 1 through Ex 3 yielding very satisfactory results.

1. An improved non-oxidative dehydrogenation process for catalytically converting an oxygen-free gaseous stream that comprises an alkane selected from ethane, propane and butane to a product stream that comprises an alkene that corresponds to the alkane by contacting the gaseous feed stream with a dehydrogenation catalyst at reaction temperature and in concurrent flow through a dehydrogenation reactor for an average contact time between the gaseous stream and the catalyst that is less than or equal to 20 seconds, wherein the improvement comprises using a supported catalyst that consists essentially of nickel, gallium and, optionally, one or more elements selected from Group VIII noble metals, alkali metals, alkaline earth metals and lanthanides, on a catalyst support.

2. The improved process of claim 1, wherein the supported catalyst has a nickel loading within a range of from greater than 25 parts by weight to less than 500 parts by weight, in each case based upon a million parts by weight of the supported catalyst.

3. The improved process of claim 1, wherein the catalyst is a catalyst subjected to calcination at a temperature of less than 750° Celsius prior to contact with the gaseous feed stream.

4. The improved process of claim 1, wherein the support is selected from alumina and alumina-silica.

5. The improved process of claim 4, wherein the catalyst support is alumina with a BET surface area within a range of from 50 square meters per gram to 150 square meters per gram.

6. The improved process of claim 1, wherein the reaction temperature lies within a range of from 570° Celsius to 750° Celsius and pressure within the dehydrogenation reactor lies within a range of from 50.7 kilopascals to 2 megapascals.

7. The improved process of claim 1, wherein the alkane is propane and the corresponding alkene is propylene.

8. The improved process of claim 1, wherein the supported catalyst has a gallium loading within a range of from 0.1 weight percent to 5 weight percent, in each case based upon weight of the supported catalyst.

9. The improved process of claim 1, wherein the supported catalyst has a alkali metal, alkaline earth metal or lanthanide loading within a range of from 0 weight percent to 5 weight percent, in each case based upon total weight of supported catalyst.

10. The improved process of claim 9, wherein the alkali metal, alkaline earth metal or lanthanide is an alkali metal selected from cesium, lithium and potassium.
